(19) United States
(12) Patent Application Publication
(10) Pub. No.: US 2010/0099096 A1
(43) Pub. Date: Apr. 22, 2010

(54) Compositions and Methods for Identifying Factors Affecting Protein Stability

(75) Inventors: Stephen Elledge, Brookline, MA (US); Hsueh-Chi Yen, Boston, MA (US)

Correspondence Address: LAW OFFICE OF MICHAEL A. SANZO, LLC, 15400 CALHOUN DR., SUITE 125, ROCKVILLE, MD, 20855, US

(73) Assignee: THE BRIGHAM AND WOMEN'S HOSPITAL, INC., Boston, MA (US)

(21) Appl. No.: 12/528,595

(22) PCT Filed: Feb. 26, 2008
(86) PCT No.: PCT/US08/02461

§ 371 Date: Aug. 25, 2009

Related U.S. Application Data
(60) Provisional application No. 60/903,826, filed on Feb. 28, 2007.

(51) Int. Cl. C12Q 1/68; C12N 15/74; C40B 40/02; G01N 33/53

(52) U.S. Cl. 435/6; 435/320.1; 506/14; 435/7.21

(57) ABSTRACT

The present invention is directed to retroviral vectors, and libraries generated from the vectors that can be used in assessing the stability of proteins and in correlating degradation with a specific E3 ubiquitin ligase. The libraries can also be used to identify factors that alter the degradation of proteins of therapeutic value and which have potential use clinically.

### CROSS REFERENCE TO RELATED APPLICATIONS

The present application claims priority to, and the benefit of, U.S. provisional application 60/903,826, filed on Feb. 28, 2007, the contents of which is hereby incorporated by reference in its entirety.

### FIELD OF THE INVENTION

The present invention is directed to compositions and methods that can be used to analyze the stability of proteins in vivo, to determine the specificity of ubiquitin ligases and to screen for factors either inhibiting or enhancing the activity of these enzymes and deubiquitinating enzymes (Dubs). In addition, the invention encompasses methods for identifying drug targets relevant to the control of particular protein degradation pathways. In the same way that the perturbation of transcriptional circuits can be used to reveal the effect of drugs on transcription using microarrays, drug signatures may be read out by their perturbation of protein stabilities on a genome-wide scale.

### BACKGROUND OF THE INVENTION

Protein degradation in eukaryotes is, in most instances, a multistep process in which ubiquitin is: a) activated by an ubiquitin activating enzyme (E1); b) transferred to the active site cysteine of a ubiquitin-conjugating enzyme (E2); and c) transferred to a lysine residue on a target protein. The final step of the process typically involves a ubiquitin ligase (E3) recognizing the specific target protein and catalyzing the transfer of ubiquitin. In many cases, ubiquitin is added to previously-conjugated ubiquitin molecules to form a polyubiquitin chain. If the chain is longer than 3 ubiquitin molecules, the tagged protein is recognized and degraded by a proteasome into small peptides. Deubiquitinating enzymes, Dubs, can reverse these ubiquitin modifications and can therefore alter protein function and stability.

Alterations in the ubiquitin system are an important contributor to a number of pathological conditions. For example, increased proteolysis through the ubiquitin-proteosome pathway is a major cause of rapid muscle wasting in fasting, metabolic acidosis, muscle denervation, kidney failure, renal cachexia, uremia, diabetes mellitus, sepsis, AIDS wasting syndrome, cancer and Cushing's syndrome (Mitch, et al., New Engl. J. Med, 335:1897-1905 (1996); Lecker, et al., J. Nutr. 129:227S-237S (1999)). In addition, ubiquitination is thought to be the method of cellular egress for a number of retroviruses, including HIV and Ebola, and there are several genetic disorders that have been associated with mutations in genes encoding E3 ligases, including Angelman syndrome, Von Hippel-Lindau syndrome and Liddle's syndrome. These associations have led to considerable interest in agents that modulate ubiquitin activity both among scientists studying disease processes and among companies developing therapeutic agents (US 20060160869; U.S. Pat. No. 6,737,244). Because of their specificity, the E3 ligase enzymes are of particular interest therapeutically.

Unfortunately, the specificity of the hundreds of E3 enzymes within cells remains largely unknown despite efforts to develop effective assays (U.S. Pat. Nos. 7,022,493; 6,740,495; 6,713,267). Until methods can be developed for associating particular enzymes with the degradation of particular proteins, the goal of developing inhibitors and enhancers of protein degradation that can be used therapeutically cannot be fully realized.

### SUMMARY OF THE INVENTION

### General Summary

The present invention is based upon the development of a system for studying the stability of mammalian proteins and for correlating the degradation of specific proteins with specific E3 ubiquitin ligases. The system utilizes retroviral vectors having sequences encoding two different marker proteins. These are both under the control of the same promoter and are separated from one another by an internal ribosome entry sequence (IRES). One of the markers serves as a standard for comparison and should preferably be a protein that does not undergo degradation as the result of the binding of an E3 ubiquitin ligase. The second marker is fused to a sequence encoding the test protein, i.e., the protein whose stability is being examined. This test protein may be encoded by a known sequence or it may be a sequence resulting from the ligation of the retroviral vector to a library of genes or open reading frames.

In a preferred embodiment, a new library of cells is created by ligating the retroviral vector sequences described above with a library of genes or open reading frames. Cells infected with these recombinant viruses express recombinant protein fusions of different stabilities. In order to assess test protein stability or associate an E3 with a particular protein or group of proteins, the cells are sorted into subgroups based upon the ratio of a second marker protein to the first marker protein (or vice versa). For example, if the first marker is dsRed (a red fluorescing protein) and the second marker is green fluorescent protein, then cells exhibiting different ratios of fluorescent absorption at red and green wavelengths can be sorted using flow cytometry. The next step is to examine the effect of a perturbation on the ratio observed in a subgroup (or series of subgroups). For example, the cells may be transformed with an RNA known to interfere with the expression of a particular E3 ubiquitin ligase or with a vector that increases the expression of an E3 ligase. If, upon reexamination, the ratio of markers has changed, this is an indication that the particular E3 ligase inhibited by the RNA or increased by the expression vector is acting upon the test protein (or test proteins) being expressed in those cells. In order to determine what particular proteins these are, the cells with the altered ratios can be sorted and the nucleic acid sequence encoding the test protein may be amplified and sequenced or, as described further below, analyzed by hybridization to a microarray of known sequences. Amplification is accomplished by the polymerase chain reaction (PCR), using primers that are based upon sequences in the retroviral vectors used to create the cells and which flank the nucleic acid encoding the protein whose stability is being assessed.

An alternative method for identifying genes encoding proteins whose stabilities change is called “Global Protein Stability Signatures by Microarray.” This method entails taking fusion library cells that are either untreated, or treated with a drug, an siRNA, or a vector that expresses a ubiquitin ligase or Dub and then sorting the differently treated cell populations into pools of defined GFP/RFP ratios. The DNA inserts from each of these pools can then by amplified by PCR and hybridized to a microarray that allows each gene to be quantified. Any gene whose abundance within a ratio pool changes its ratio in response to the treatment and enters a different ratio pool will be identified. In this way entire libraries can be screened in one experiment.

The systems described above can also be used to screen test compounds or RNAi libraries for their affect on the degradation of a known protein. In this case, rather than create an entire library, the second marker sequence may be fused to a single sequence encoding the protein of interest. The vector is then introduced into cells and these are sorted as described previously. The test compounds or interfering RNAs are then incubated with the cells to determine if they alter the ratio of markers. In cases where the known protein has therapeutic value, this procedure provides a means for identifying new drug candidates.

### Specific Aspects of the Invention

In its first aspect, the invention is directed to a retroviral vector (e.g., a Lentiviral vector) comprising: a promoter; a sequence encoding a first marker protein lying 3′ to the promoter; an internal ribosome entry sequence (IRES) lying 3′ to the sequence encoding the first marker protein; a sequence encoding a second marker protein that is different from the first marker protein and that lies 3′ to the internal ribosome entry sequence; and a sequence encoding a test protein lying 3′ to the sequence encoding the second marker protein. The sequence encoding the second marker protein and the sequence encoding the test protein should be adjacent to one another so that they form a single fusion protein when expressed. Both the sequence encoding the first marker protein and the sequence encoding the second marker protein are operably linked to the promoter, i.e., transcription of the marker protein sequences is under the control of the promoter and the transcripts produced are correctly translated into the desired proteins. Any promoter that is active in mammalian cells may be used, with the most preferred being the human cytomegalovirus immediate early promoter (CMV). The preferred marker proteins are dsRed or green fluorescent protein (GFP). However, other fluorescent marker proteins can also be used provided that they absorb at wavelengths that can be distinguished from one another.

As an alternative to constructing a retroviral library based upon an existing open reading frame library or gene library, a retroviral gene trap vector can be constructed in which there is a marker1-IRES-marker2 cassette that is followed by a splice donor, where marker1 and marker2 are two different marker proteins that can be distinguished from one another during experiments, preferably dsRed and GFP. Once introduced into cells, the vector will insert by retroviral integrase-mediated non-homologous recombination upstream of endogenous genes and regulate expression by splicing into existing exons to make gene fusions. Gene fusions will make protein fusions one third of the time on average. Retroviruses with three different reading frames will allow capture of all possible exons. Infection should be designed so that there is only about one vector inserted in each cell and the cells can then be sorted and analyzed as described above.

The invention also includes eukaryotic cell libraries comprising the retrovirus vectors or the retroviral gene trap vectors described above and subgroups derived from the libraries that have cells that exhibit the same ratio with respect to marker proteins. Many different types of cells may be used in making these libraries including 293T cells; NIH-3T3 cell, CHO cells, HeLA cells, and a LM(tk-) cells. The most preferred of these is 293T cells.

In another aspect the invention is directed to a method of determining whether a test compound alters the stability of recombinant proteins, e.g., the test proteins described above and to the identification of the proteins of altered stability. The method entails first culturing cells that have been grouped together based upon having the same ratio of marker proteins. Culturing is carried out in the presence of the test compound or after introducing a nucleic acid encoding the test compound into the cells. The ratio of second marker protein to first marker protein is then determined and a comparison is made between this ratio and the ratio obtained when the cells are cultured in the absence of test compound. For example, the Global Protein Stability Signatures by Microarray method can be used to determine which proteins move to pools of altered stability. Using this comparison, it may be concluded that the stability of particular recombinant proteins have been altered if the ratio determined in the presence of the test compound is different than the ratio determined in the absence of the test compound. In cases where the test compound is either an E3 ubiquitin ligase or an siRNA that interferes with the expression of an E3 ubiquitin ligase, this method can be used to associate a particular E3 enzyme with a specific protein.

The method can also be adapted to the screening of test compounds for their effect upon the degradation of a known protein. In this case, the protein being examined would be the test protein, cells would be sorted based upon protein stability and incubations would be performed using the test compounds. In instances where a test compound either contributed to a disease state or had a therapeutic effect, the assay could be used to identify compounds of potential clinical value.

### DETAILED DESCRIPTION OF THE INVENTION

### A. General Description

The present invention is directed to an assay system having several components. The first component is a reporter library of mammalian cells in which each cell contains a retrovirus expressing dsRed followed by an internal ribosome entry sequence (IRES) and a sequence coding for a protein in which GFP is fused to a unique protein (protein X). All of these components are under the control of a single promoter, preferably a CMV promoter. Because each cell expresses a single GFP fusion with a specific stability dictated by the fusion protein, it will have a defined GFP/RFP ratio in which the turnover rate of the GFP-fusion is standardized to the turnover rate of RFP. This master library may be sorted based on the GFP/dsRed ratio into sublibrary pools of common ratios that reflect the stability of the GFP-protein X fusion relative to dsRed. The use of a common promoter, dual reporters on the same transcript and the sorting into pools of constant ratios prevents expression levels due to differing integration sites to alter the read out. It will be recognized that detectable marker proteins other that dsRed and GFP can also be used, with the only essential requirement being that the two markers be distinguishable from one another.

In addition to the production of cell libraries as described above, a method must be available for sorting cells into pools with a similar ratio of markers. This can be accomplished, for example, using single-cell sorting by flow-cytometry. Once a group of such cells has been obtained, flow cytometry, or a comparable technique, can be used to determined whether protein stability has been altered in response to a given perturbation and to isolate those cells exhibiting stability changes. It should be noted that proteins with cell cycle regulated stabilities will sort into a pool based on their ratio at the time of sorting, but will change afterwards. This problem can be eliminated by sorting the population twice for altered ratios, once before perturbation and once afterwards to find the genes that differ only in the perturbed pool. Alternatively, cell cycle regulated proteins can be determined by sorting the library for both GFP/dsRed ratios and DNA content to determine if a protein displays different stabilities depending upon which stage of the cell cycle it is in.

If desired, the DNA encoding various test proteins within a pool of defined stability may be analyzed directly, e.g. using a microarray of known sequences. Using such methodology, one can determine which proteins are relatively stable or rapidly degraded within a given population and compare the results obtained to other populations. For example, the differences between the stability of proteins in pathological cells and their normal counterparts may provide important information concerning disease processes. Alternatively, as suggested above, a pool of cells exhibiting similar protein stability may be perturbed in some manner. Perturbations may include, for example, proteasome inhibition or inhibition of the Cul1 pathway. Other types of perturbations include the use of RNAi against specific E3s or Dubs, growth factors, oxidative stress and factors causing DNA damage. In addition vectors increasing the expression of particular E3s may be used.

Finally, there must be a method for identifying the specific cloned sequences that are present in the cells within a selected pool. Because each gene encoding a test protein is within a retroviral sequence, it is possible to recover genes by PCR in order to identify the protein whose levels are altered by a particular perturbation. Identification may involve direct sequencing or a group of genes within a pool can be analyzed by hybridization to a microarray of known sequences. For example, cells expressing a rapidly degraded protein (low GFP/RFP ratio) may be sorted into a pool of a much longer half-life upon a perturbation that specifically affects turnover of the particular GFP-fusion in that cell while cells where the perturbation has no effect on the half-life of the GFP-fusion will remain in the same cell pool. Thus, microarray analysis of all pools in a perturbation experiment can identify all GFP-fusions that have been stabilized by that particular perturbation.

In cases where the test protein is known and either enhancing or inhibiting its expression may be of therapeutic value, the system can be used to screen for new drug candidates. However, the method is not limited to examining the effects of increasing or reducing the levels of E3s or Dubs, any protein can affect the stabilities of other proteins if it controls a Dub or E3 ligase pathway. Therefore, new functions of proteins can be identified by examining their effects on global protein stabilities.

### B. Specific Aspects of the Invention

Genetic Reporters of Protein Turnover

A critical requirement for the system described herein is that one be able to isolate mammalian cell libraries that stably express specific transcription units, e.g., DsRed-IRES-GFP-Gene X (where “Gene X” encodes the test protein, i.e., “protein X”), under conditions where the ratio of markers, e.g., DsRed to GFP, is stable. Moreover, it is critical that one be able to identify cells that have undergone changes in the ratio of markers. We have found that while cells infected with the Retro-DsRed-IRES-GFP virus display a range of absolute levels, reflecting the sites of integration, the GFP/DsRed ratio forms a homogenous peak over all the cells analyzed for a given clone. Importantly, analysis of a doxycycline inducible DsRed-IRES-GFP expressing cells over a range of doxycycline levels indicates that the GFP/DsRed ratio does not vary, despite the fact that the total GFP or DsRed levels increase with doxycycline.

Another important feature of this system is that it be able to accurately detect differences in half-lives between different proteins fused to the marker, GFP in our experiments. To test this, we fused the D1 (t_{1/2}=1 h) and D4 (t_{1/2}=4 h) degrons (derived from ornithine decarboxylase) that impart different stabilities upon GFP (t_{1/2}=24 h) and examined both the half-life of the GFP-fusion protein and the GFP/DsRed ratio. We found that GFP fusions displaying differences in half-lives from 1 to 4 hours or 4 to 24 hours can be easily distinguished based on the GFP/DsRed ratio. Moreover, addition of proteasome inhibitor (MG132) dramatically stabilizes both the GFP-D1 and GFP-D4 proteins, altering the GFP/DsRed ratio consistently from a low GFP/DsRed ratio to a high ratio.

To examine whether this system could be extended to proteins that are known to be degraded through phosphorylation-dependent SCF pathways, GFP-Cdc25A and GFP-cyclin E fusions were made. Cdc25A degradation occurs through Chk1-mediated phosphorylation and requires SCF^{β-TRCP} while cyclin E degradation involves phosphorylation of T380 and involves the SCF^{Fbw7} complex. Addition of MG132 to cells stably expressing the Cdc25A fusion increases the GFP/DsRed ratio. Likewise, mutation of T380 in cyclin E to alanine increases the GFP/DsRed ratio. Thus, such reporters are sensitive to mutations that affect the turnover of the protein.

Development of Novel Reporter Cell Collections

It will be recognized by those of skill in the art that many approaches may be used for making libraries of cells expressing DsRed-IRES-GFP fusion proteins (or comparable sequences) with a large number of human genes. For example, we have successfully generated a retroviral library in which an 8000 gene human ORFEOME collection was fused to GFP using the Gateway system and integrated this into 293T cells. In this setting, ORFs are fused to GFP downstream of the DsRed-IRES cassette. In an alterative approach, we generated a retroviral gene trap vector (based on a previously described ERM Enhanced Retroviral Mutagenesis vector) in which the DsRed-IRES-GFP cassette is followed by a splice donor. Random integration of this vector into protein coding genes produces a GFP protein fused to portions of the integrated gene 3′ to the integration site by splicing to the appropriate downstream splice acceptor. We have demonstrated that this vector efficiently integrates into protein coding genes, as indicated by our ability to generate many different protein localization patterns as a result of fusion with different proteins with distinct localization properties. Using this vector, it is possible to generate libraries of cell lines containing hundreds of thousands of independent integration events, potentially targeting a large fraction of the genes in the genome.

Proof of Principle Experiments

Using the materials and procedures described above, we have found that it is possible to generate suitable cellular libraries and to sort these into populations of cells which display distinct ratios of GFP/DsRed. Moreover, we have found that the GFP/DsRed ratio is stable over a 1 month period of culture. This is critical because it provides sufficient time for cells to be manipulated and sorted for alterations in the GFP/DsRed ratio.

As a proof of principle, cells stably expressing DsRed-IRES-GFP-Cdc25A were mixed with a population of EGFP library cells in a ratio of 1 to 1000. A plasmid expressing a Cul1 dominant negative mutant protein was introduced by transfection and cells were sorted for altered GFP/dsRed ratios. Cells from the presorted and post-sorted populations were subjected to PCR to determine the enrichment in cells containing the Cdc25A transgene. We found more than a 200-fold enrichment for cells containing the Cdc25A transgene, indicating that this approach has the potential to strongly enrich for substrates of the SCF.

In a preliminary pilot screen employing a small population of cells expressing ORFEOME clones, we identified several clones, including the p21 gene, as being induced when cells are transfected with the Cul1 dominant negative vector. Cul1 is an essential component of the SCF ubiquitin ligase family. A dominant negative Cul1 interferes will all of the SCF ligase family members. p21 has been reported to be ubiquitinated by the SCF^{Skp2} complex. Thus, this approach has the potential to allow the identification of proteins whose abundance changes in response to particular perturbations.

### EXAMPLES

### Example 1

Determination of Protein Stability

In order to test whether GFP/RFP ratios can be used to distinguish proteins of different half-lives, we created cell lines that express RFP-IRES-GFP fusions with degrons conferring different half-lives (1 h, 4, and 24 h) fused to GFP. Cells stably expressing these fusions displayed distinct GFP/RFP ratios in the order of their stability (the short-lived GFP degron fusion had the lowest GFP/RFP ratio). We then generated an RFP-IRES-GFP-p53 fusion and integrated this construct into 3 different cell lines, each having a distinct p53 turnover pathway. When overlaid onto the GFP/RFP ratios determined for the GFP-degron series, it is evident that the p53 half-life can be determined from its position in the overlay and that the half-life of p53 varies according to the genetic background of the cell. U205 cells have an intact p53 degradation pathway and p53 turnover in this cell type parallels that of the 1 hour GFP degron. Hela cells express the E6 protein, which promotes E6-AP-dependent p53 degradation. In these cells, the GFP-p53/RFP ratio is lower than that seen in U205, indicating that p53 is more unstable in these cells. In contrast, the GFP-p53 fusion is highly stable in 293T cells, which express T-antigen (which binds and sequesters p53). The results obtained indicate that the GFP/RFP ratio serves as an accurate measure of the relative half-lives of proteins encoded in the RFP-IRES-GFP-fusion cassette. As described further below, we have also found that the SCF substrate Cdc25A can be analyzed using this system.

Using an ORFEOME (initially 8,000 clones with 4,000 clones added more recently), we have generated a library of 12,000 RFP-IRES-GFP-gene fusions in a retroviral vector and have created a library of cell lines expressing these GFP fusions (100-fold coverage). As expected, there is a wide distribution of GFP/RFP ratios within the library, as determined by flow cytometry. However, we have found that we can sort these cells into distinct pools based on these ratios and that the GFP/RFP ratio for each pool is stable for more than 40 days in culture, providing sufficient time for performing experiments.

### Example 2

Detection of Unstable Proteins Using Microarrays

In order to be able to rapidly identify genes whose abundance is altered in response to a perturbation or to simply determine the half-life of the proteome, we developed microarrays which specifically detect an ORFEOME. Using primer pairs that are specific to the retrovirus used to deliver the GFP-fusion, we can amplify sequences by PCR and analyze the genes present in each pool by direct hybridization. This has now been done across 7 pools in the absence of perturbations to identify unstable proteins and has also been done with and without addition of the proteasome inhibitor MG132 to identify proteins whose degradation requires the proteasome. In some cases, the same gene is represented in multiple pools but perturbation shifts the peak pool to a higher GFP/RFP ratio.

In order to determine whether we can use cell library pools in conjunction with microarrays to determine the relative half-lives of proteins, we randomly chose 96 genes across the 7 pools and cherrypicked individual ORFs from our arrayed ORFEOME collection. Each ORF was recombined into the RFP-IRES-GFP vector separately, virus produced, and used to transduce cells independently. We then measured the GFP/RFP ratio for each clone and compared it with the GFP/RFP ratios for a GFP-degron series in order to obtain an approximate half-life. The turnover rates for these genes matched the rates initially determined by the pools they were isolated from and moreover, it was possible to determine a relative half-life based on where the individual clone fell on the GFP/RFP ratio plot for the GFP-degron series. These data indicate that the approach provides a robust read-out for protein half-life and reveals that microarrays can be used to identify genes whose encoded protein displays a particular half-life.

### Example 3

Proteins Whose Abundance is Regulated by the Proteasome

We sorted a HeLa GFP-fusion library in the presence and absence of a proteasome inhibitor (MG132) and compared the genes in each pool by comparative hybridization. This resulted in a shift of a substantial number of cells to higher GFP/RFP ratios and each of these changes could be tracked through comparative analysis of quantitative microarrays. Two dozen genes displaying the highest degree of change in the GFP/RFP ratio with MG132 were selected, individually cloned into the RFP-IRES-GFP vector and cell lines generated. The GFP/RFP ratio was determined for each gene in the presence and absence of MG132. In every case examined, there was a change in the GFP/RFP ratio discernable by flow-cytometry upon addition of proteasome inhibitor. To demonstrate that stabilization by MG132 was independent of the GFP fusion, eleven of these genes were cloned into an HA-tagged vector and the steady-state abundance of the tagged protein determined by immunoblotting in the presence and absence of MG132. This collection includes both rapidly and slowly turned over proteins. In all cases, an increase in the abundance of the tagged protein was observed upon addition of MG132. These data indicate that the system is capable of identifying proteins whose abundance increases when its degradation pathway is inhibited.

All references cited herein are fully incorporated by reference in their entirety. Having now fully described the invention, it will be understood by those of skill in the art that the invention may be practiced within a wide and equivalent range of conditions, parameters and the like, without affecting the spirit or scope of the invention or any embodiment thereof.

1. A retrovirus vector comprising:
a) a promoter;
b) a sequence encoding a first marker protein lying 3′ to said promoter;
c) an internal ribosome entry sequence (IRES) lying 3′ to said sequence encoding said first marker protein;
d) a sequence encoding a second marker protein, wherein said second marker protein is different from said first marker protein and lies 3′ to said internal ribosome entry sequence;
e) a sequence encoding a test protein lying 3′ to said sequence encoding said second marker protein;
and wherein both the sequence encoding said first marker protein and the sequence encoding said second marker protein are operably linked to said promoter.

2. The retrovirus vector of claim 1, wherein said retrovirus is a Lentivirus.

3. The retrovirus vector of claim 1, wherein said promoter is the CMV promoter.

4. The retrovirus vector of claim 3, wherein said first marker protein is either dsRed or green fluorescent protein (GFP).

5. The retrovirus vector of claim 4, wherein said second marker protein is either dsRed or GFP.

6. The retrovirus vector of claim 5, wherein said test protein is derived from a library of human open reading frames.

7. A retroviral gene trap vector in which a DsRed-IRES-GFP cassette is followed by a splice donor.

8-17. (canceled)

18. A eukaryotic cell library comprising either:
a) a retrovirus vector comprising:
i) a promoter;
ii) a sequence encoding a first marker protein lying 3′ to said promoter;
iii) an internal ribosome entry sequence (IRES) lying 3′ to said sequence encoding said first marker protein;
iv) a sequence encoding a second marker protein, wherein said second marker protein is different from said first marker protein and lies 3′ to said internal ribosome entry sequence;
v) a sequence encoding a test protein lying 3′ to said sequence encoding said second marker protein;
and wherein both the sequence encoding said first marker protein and the sequence encoding said second marker protein are operably linked to said promoter; or
b) a retroviral gene trap vector in which a DsRed-IRES-GFP cassette is followed by a splice donor.

19. The eukaryotic cell library of claim 18, wherein said retrovirus is a Lentivirus.

20. The eukaryotic cell library of claim 18, wherein said promoter is the CMV promoter.

21. The retrovirus vector of claim 3, wherein said first marker protein is either dsRed or green fluorescent protein (GFP).

22. The eukaryotic cell library of claim 18, wherein said test protein is derived from a library of human open reading frames.

23. The eukaryotic cell library of claim 18, wherein the cells in said library are 293T cells.

24. The eukaryotic cell library of claim 18, wherein the cells in said library are selected from the group consisting of: NIH-3T3 cells, CHO cells, HeLA cells, and a LM (tk-) cells.

25. The eukaryotic cell library of claim 18, wherein said eukaryotic cell library has into populations based upon the ratio of said second marker protein to said first marker protein.

26. A method of determining whether a test compound alters the stability of a recombinant protein, comprising:
a) culturing cells of the library of cells of claim 18 in the presence of said test compound or introducing a nucleic acid encoding said test compound into said cells;
b) determining the ratio of said second marker protein to said first marker protein;
c) comparing the ratio determined in step b) with the ratio determined for cells of the same sublibrary of cells cultured in the absence of said test compound; and
d) concluding that the stability of said recombinant protein has been altered if the comparison of step c) indicates that the ratio determined for cells in the presence of said test compound is different than the ratio determined for cells in the absence of said test compound.

27. The method of claim 26, further comprising amplifying a nucleic acid sequence encoding said recombinant protein in said cells using the polymerase chain reaction (PCR).

28. The method of claim 27, further comprising determining the identity of said nucleic acid sequence comprising hybridizing said nucleic acid sequence to a microarray of known sequences.

29. The method of claim 27, wherein said test compound is either an E3 ubiquitin ligase or an RNA that interferes with the expression of an E3 ubiquitin ligase.
